(19) United States
(12) Patent Application Publication
(10) Pub. No.: US 2014/0257483 A1
(43) Pub. Date: Sep. 11, 2014

(54) METHOD AND DEVICE FOR CERVICAL BONE MARROW ASPIRATION FOR USE IN AN ANTERIOR CERVICAL DISCECTOMY AND FUSION PROCEDURE

(71) Applicant: Karl W. Swann, San Antonio, TX (US)

(72) Inventor: Karl W. Swann, San Antonio, TX (US)

(21) Appl. No.: 13/785,460

(22) Filed: Mar. 5, 2013

(51) Int. Cl. A61F 2/44; A61B 10/02

(52) U.S. Cl. CPC A61F 2/4455; A61B 10/025
USPC 623/17.11

(57) ABSTRACT

Applicant provides a surgical method related to ACDF and a device for practicing the surgical method. The device comprises an aspiration needle assembly having an aspiration needle, the aspiration needle assembly adapted for receipt into a bony body at a preselected depth. Applicant's surgical method is an anterior cervical discectomy and fusion but one in which bone marrow is aspirated from a selected cervical vertebral body, rather than the iliac crest or other area of a patient's body. Moreover, Applicant has discovered that orienting the axis of the needle and the location of the needle such that the needle draws bone marrow from adjacent end plate rather than a center of the cervical vertebral body provides a richer bone marrow concentration, that is to say, richer in stem cells.

### FIELD OF THE INVENTION

Medical operations and devices therefor, namely, a method and device for cervical bone marrow aspiration in an anterior cervical discectomy and fusion procedure.

### BACKGROUND OF THE INVENTION

Anterior cervical discectomy and fusion (ACDF) is a surgical procedure performed to remove a herniated or degenerative disc in the cervical (neck) spine. The surgeon approaches the spine from the front, through the throat area. The surgeon moves aside the neck muscles, trachea, and esophagus, and the disc and bony vertebrae are accessed. After the disc is removed, the space between the bony vertebrae is empty. The surgeon fills the open space with a bone graft or a device, such as a cage and bone graft fusion assembly. The graft/assembly serves to fuse the two adjacent vertebrae to create a spinal fusion and stability.

### SUMMARY OF THE INVENTION

A surgical method is disclosed including the steps of providing a bone marrow aspiration needle assembly and providing a bone graft/assembly. The surgical site is identified and exposed, and includes at least a portion of an anterior vertebrae. Using a bone marrow aspiration needle, bone marrow from a selected cervical vertebrae body is aspirated. At least one cervical disc is removed. The vertebrae adjacent the removed disc is prepared. A bone marrow concentrate from the aspirated vertebral bone marrow is prepared. The bone marrow concentrate is applied to the bone graft or the fusion assembly. The bone graft or fusion assembly is inserted into the inter-vertebrae space. The vertebrae adjacent the inserted bone graft/assembly may be fixed.

Applicant provides a method for anterior cervical disc removal and vertebrae stabilization using aspiration and, in one embodiment, an aspiration needle similar to that set forth herein. Unlike other ACDF procedures, the aspiration is performed on the vertebrae at the operation site and the bone marrow concentrate is enriched. The enriched concentrate, being rich in stem cells, is applied to the bone graft or bone graft and cage fusion assembly, and bone graft/assembly is then inserted into the empty inter vertebrae space.

### BRIEF DESCRIPTION OF THE DRAWINGS

FIG. 1 is an illustration of a site for practicing Applicant's method.

FIG. 2 is a perspective view of a needle assembly for use with Applicant's method.

FIG. 3 is a perspective view of a fusion assembly including a cage and a bone graft for use with Applicant's method.

FIG. 3A is a perspective view of a cage for use in Applicant's method

FIG. 3B illustrates a step in which a spine needle is used in Applicant's method.

FIG. 3C is an elevational view of the fusion assembly.

FIG. 4 illustrates the insertion of the aspiration needle into a selected aspiration site.

FIG. 4A illustrates the aspiration needle penetration depth adjustment step of Applicant's method.

FIG. 5 illustrates the removal of a stylet from the aspiration needle assembly after the needle has been inserted all the way into the selected vertebral body site.

FIG. 6 illustrates the aspiration of the bone marrow at the selected site through the aspiration needle assembly.

FIG. 6A illustrates a preferred orientation of the needle with respect to the plane of the end plate.

FIGS. 6B and 6C illustrate preferred target areas on cervical vertebrae bodies for removing bone marrow.

FIG. 7 illustrates the transfer of the bone marrow aspirate into a collection cup.

FIG. 8 illustrates the aspiration cup with the bone marrow aspirate being centrifuged.

FIG. 9 illustrates the centrifuge separated bone marrow aspirate including the bone marrow concentrate.

FIG. 10 illustrates the removal of the bone marrow concentrate from the collection cup.

FIG. 11 illustrates the fusion assemblies prior to hydration/saturation.

FIG. 12 illustrates the hydration/saturation of the fusion assemblies in a condition in which they are ready to be inserted into the vertebral space created by the removed discs.

FIGS. 13A, 13B, and 13C illustrate the use of an inserter for placing the BMC soaked fusion assembly into the prepared intervertebral space.

FIG. 14 shows the (optional) fixation step.

### DETAILED DESCRIPTION OF THE PREFERRED EMBODIMENT

This application incorporates by reference U.S. patent application Ser. No. 13/323,269, filed Dec. 12, 2011.

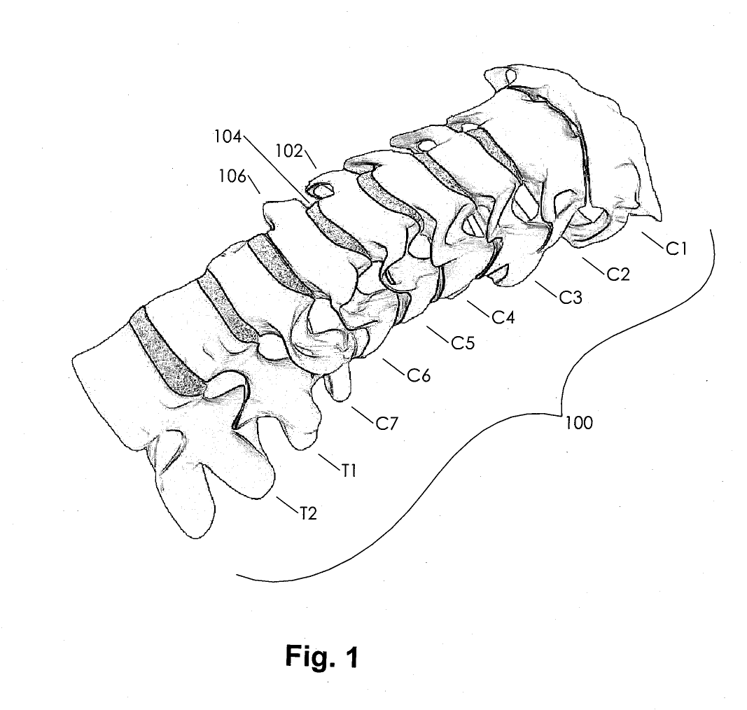

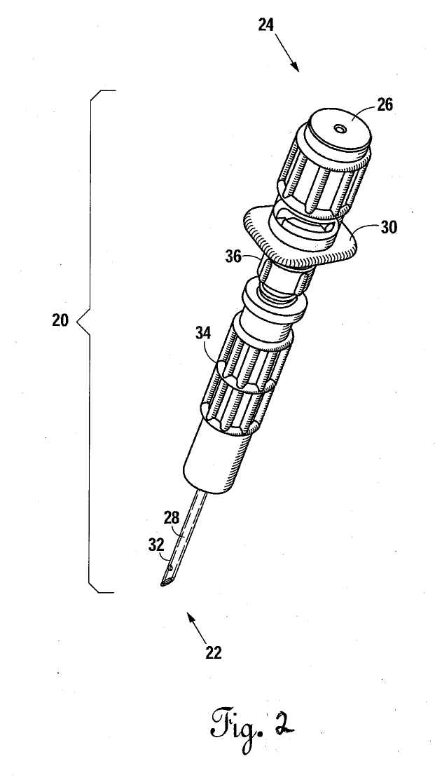

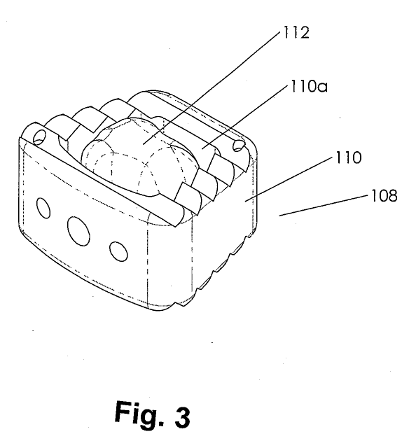

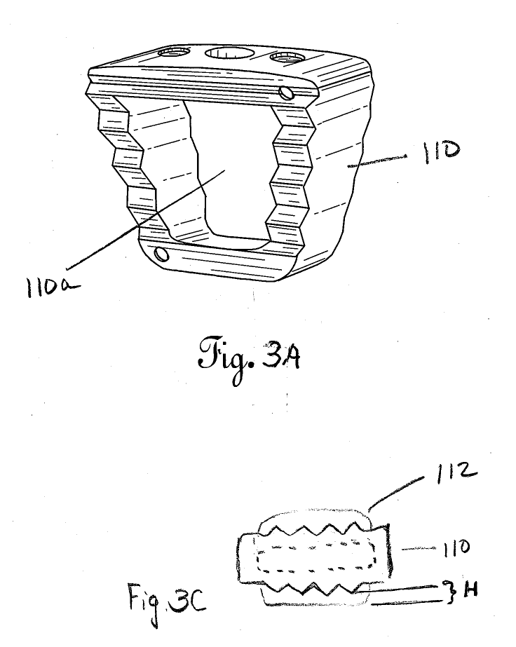

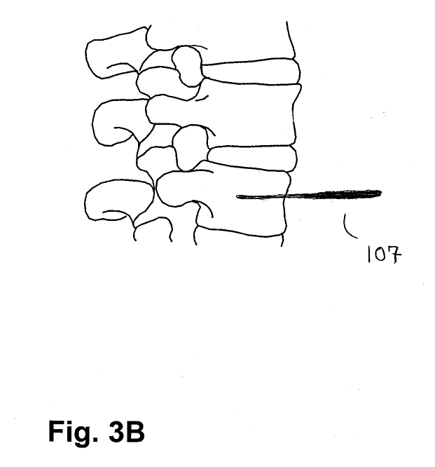

Using conventional diagnostic methods and procedures, the degenerative or bulging discs which are to be removed are identified according to their location on the vertebral column. In FIG. 1, a section or site 100 of vertebrae C-1 through T-2 are illustrated, as a preferred site for Applicant's method. The patient is brought into the operating room and placed face up on the table with the neck exposed and the patient anesthetized. Once asleep, their neck is cleaned and prepared, and the surgical instruments, including the aspiration needle assembly 20, a cage 110, and a bone graft 112 is provided (see FIGS. 2, 3, 3A).

The site is exposed using conventional ACDF surgical procedures, which typically include retraction of the esophagus, throat, and carotid artery. An incision about 2 to 3 inches is made on the neck and the muscles in the neck moved aside with the trachea, esophagus and arteries retracted. The muscles that support the front of the spine are lifted and held aside so the surgeon can clearly see the bony vertebrae and the discs.

The aspiration portion of the surgical technique described herein includes preparing the needle, introducing the needle, advancing the needle, removing the stylet, screwing syringe into hub, aspirating the bone marrow, and discharging the syringe into a concentrate cup.

The aspiration site will typically be one or more of the vertebral bodies to either side of one of the affected discs. If there are two discs selected for removal, typically the aspiration site will be the vertebral body between the two and, if there are three discs to be removed, the surgeon will typically choose the one most readily accessible, typically a vertebral body between the upper and lower of the affected discs. Here, FIG. 1 illustrates degenerative disc 104 selected for removal. Vertebral bodies 102/106 are adjacent this disc and the most accessible of these is typically used for aspiration.

Next, at the site 100, a vertebral body for aspiration is confirmed. This is typically done by inserting a spine needle 107 or other suitable device and then x-raying as with as with a fluoroscope (see FIG. 3B), mounted on a “C arm” to check and confirm that the aspiration site is a vertebral body adjacent to one of the selected discs. At this point, the surgeon may confirm using fluoroscopy (real time x-ray) that the spine needle is in the proper cervical site for disc removal.

The aspiration apparatus 20 is then used. Before aspiration, remove and reinsert the stylus 28 one time to assure that it can be easily separated from the cannula of the needle 32 once it is inserted into the bone. To remove the stylet, it may be rotated 90° counterclockwise and pulled straight out. Replace the stylet in the aspiration apparatus 20 and lock it back in place by rotating it 90° clockwise so that it is aligned with the slot in the lower portion of the stylet cap. Remove the plastic guard from the aspiration needle 32.

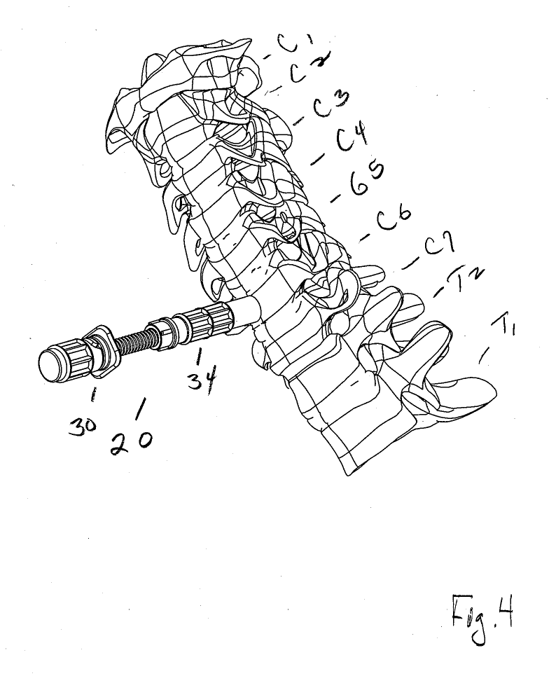

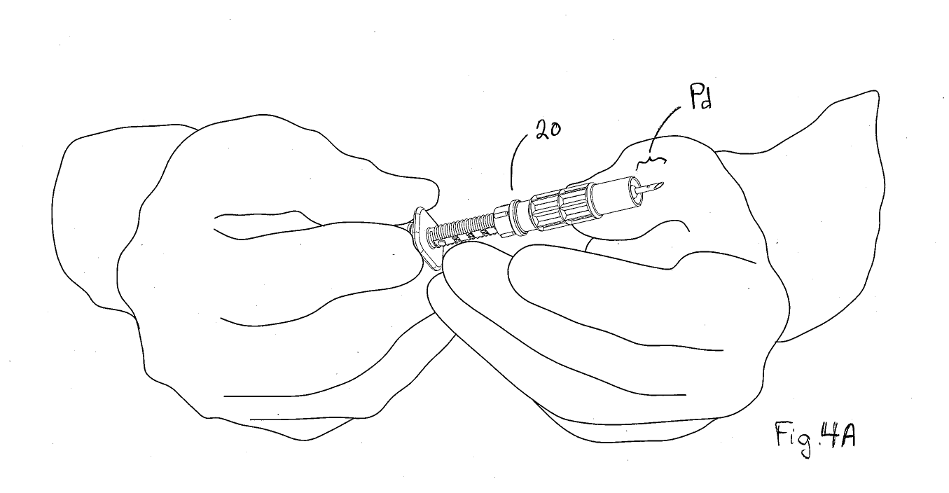

Next, the aspiration assembly 20 is prepared with a selected penetration depth Pd (see FIG. 4A). The preceding step in which a spine needle is inserted may be used to help the surgeon select the proper needle depth. The depth may also be set from the surgeon's experience and judgment, and according to the physiological characteristics, age, sex, physical condition, etc., of the patient. A typical penetration depth Pd, which is the distance between the removed end of the needle and the removed end of the needle guard, is typically about 18 to 14 millimeters (preferred about 10 to 12 millimeters). This is typically sufficient to penetrate the cortex of the vertebrae and enter the bone marrow cavity.

Adjust the depth of the needle by threading the depth guard 34 up or down based on the individual patient's anatomy and lock the lock nut 36 in place by spinning it down in a clockwise direction to finger tight. If depth needs adjustment, unlock the locking nut by spinning the nut upward in a counterclockwise direction to its starting position at the top of the needle. Grasp the aspiration assembly 32 and introduce it through the prepared incision into the selected body with the trajectory determined by the approach and the anatomical site, and a target area for aspiration as set forth herein.

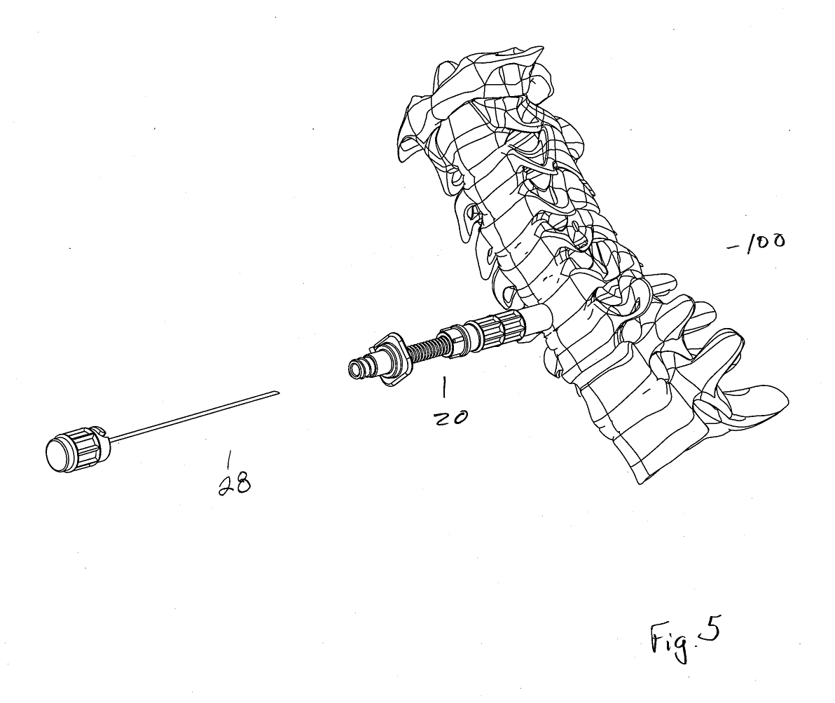

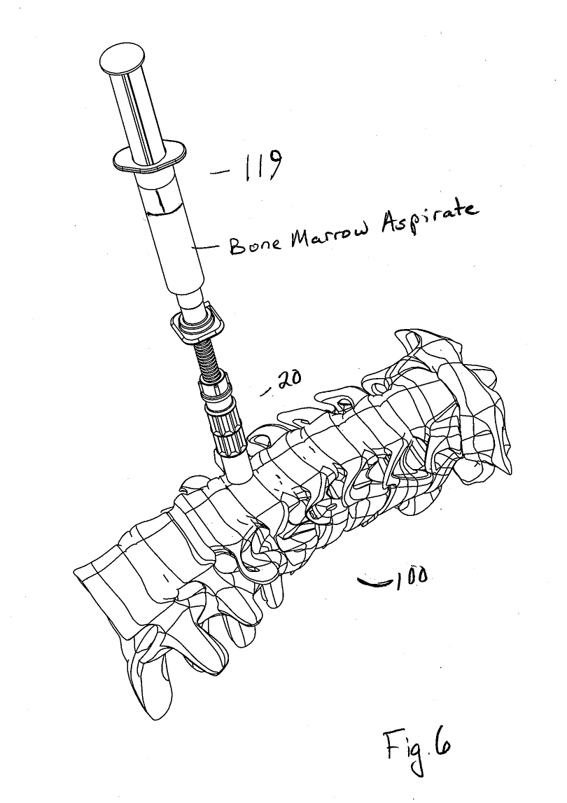

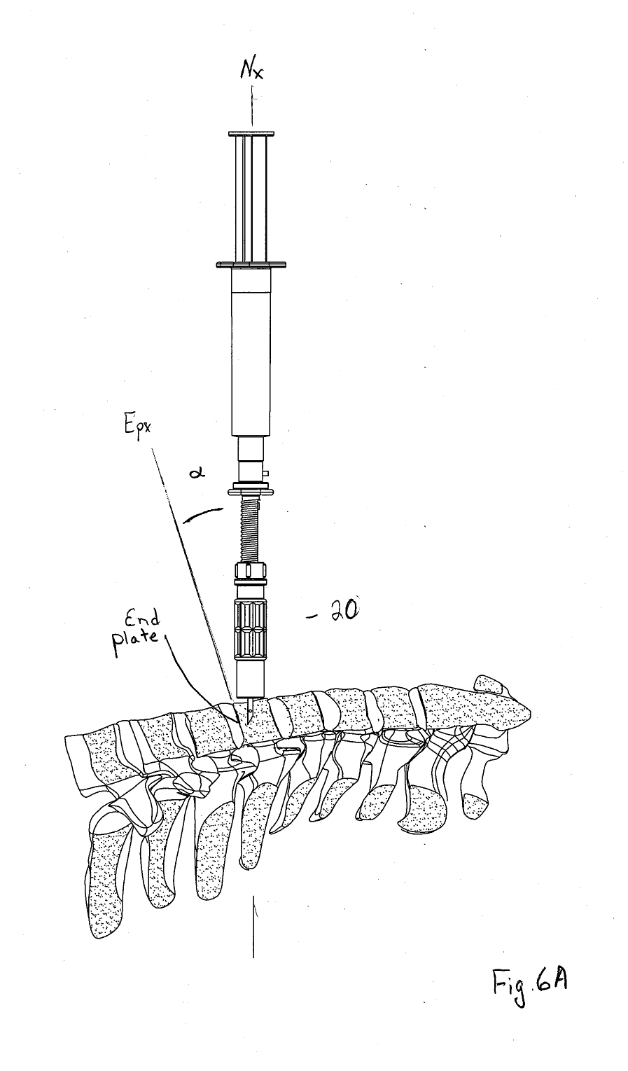

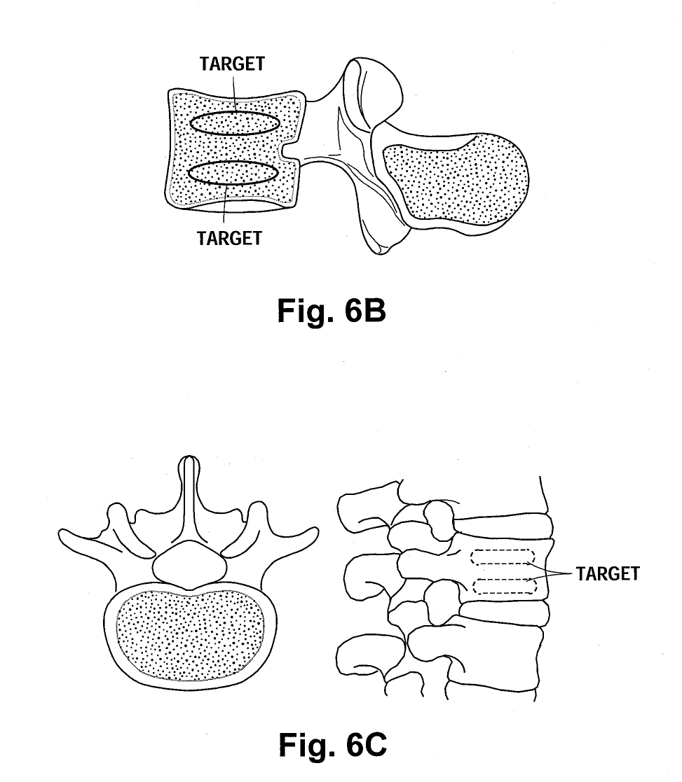

The needle is introduced into the vertebral body, typically about midpoint between the lateral surfaces and the top and bottom surfaces (see FIG. 4). From an axis or plane of the end plate (see FIG. 6A), an angle of between about 5° to 40°, or 10°-30° preferred about 20°-30° is provided to get close to one of the selected end plates. FIGS. 6B and 6C show these target areas. Applicants believe aspirate from this location is richer in stem cells than aspirate further from an end plate.

Advance the needle assembly using gentle but firm pressure. Rotate the assembly in alternating clock and counterclockwise motion or gently tap the needle using a mallet. Decreased resistance may be encountered upon penetration of the cortex and entrance of the bone marrow cavity of the vertebral body. Stylus 28 is then removed (see FIG. 5).

A syringe 119 is connected to the aspiration needle and about 2 to 10 milliliters of bone marrow is aspirated into the syringe chamber (see FIG. 6). If the bone marrow does not aspirate easily, the needle may be repositioned slightly by rotating the needle either clockwise or counterclockwise or advancing the needle further into the vertebral body using fluoroscopy. If bone marrow still does not aspirate, remove the syringe from the needle, remove the needle from the incision, reinsert the stylus, and repeat the steps above with a slight change in needle trajectory or change the penetration depth.

Applicants have discovered that the closer the aspiration is taken to the end plates of the vertebral body, the higher the yield of stem cells in the concentrate. End plates are the top and bottom portions of the vertebral body that interface the discs. The end plate is composed of a layer of thickened cancellous bone. This is where new bone is being formed. Therefore, the axis of the aspiration needle may be directed, along with the depth of the needle, so as to place the removed end of the needle such that it is aspirating closer to one of the end plates in a Target Area (see FIGS. 6B and 6C). This angle α may be about 10 to 30 degrees from perpendicular (see FIG. 6A).

Bone marrow concentrate (BMC) uses the body's ability to heal itself by using regenerative stem cells found in a patient's own bone marrow. Bone marrow is the soft tissue found in the hollow interior of bones. In adults, marrow contains a rich reservoir that produces new blood cells, including regenerative cells. After centrifuging, the concentrate is 5 to 11 times richer in regenerative stem cells and growth factors. Autologous adult stem cells from the ACDF operation site are employed to facilitate spinal fusion and help produce graft site morbidity.

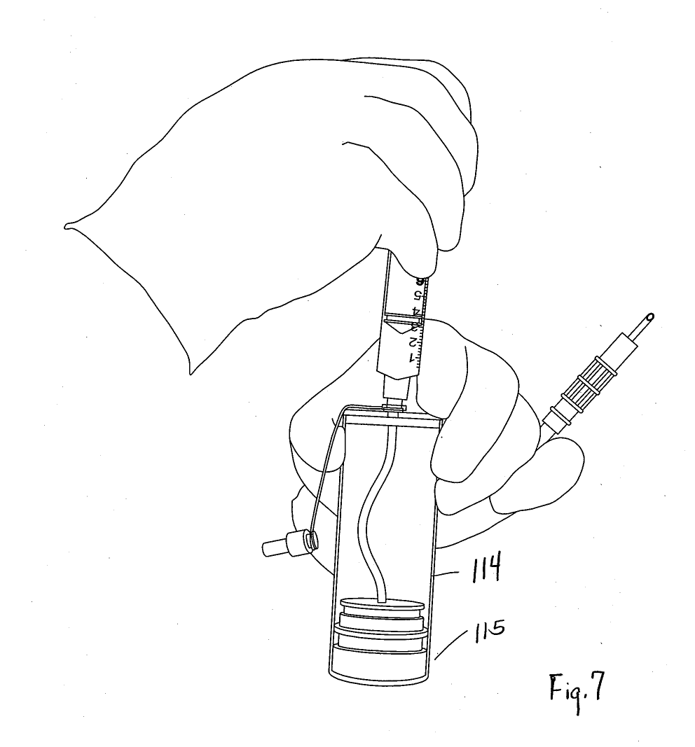

Following aspiration, transfer the syringe containing the aspirated bone marrow into a Cyclonic cup 114 (see FIG. 7) or other suitable device and dispose the needle in accordance with applicable hospital procedure. The aspiration site may be sealed with a suitable hemostatic agent, such as bone wax. The Cyclone System is complete sterile and ensures that the concentration delivered to the patient is consistent and has high viable stem cells without waves (plasma/red blood cells).

Following the aspiration step, the disc may be incised and removed in a conventional manner. The disc space is prepared using standard discectomy instruments and technique for an anterior cervical discectomy and fusion procedure. Sufficient cleaning and roughing of the end plates is important at this step for vascular supply to the bone graft.

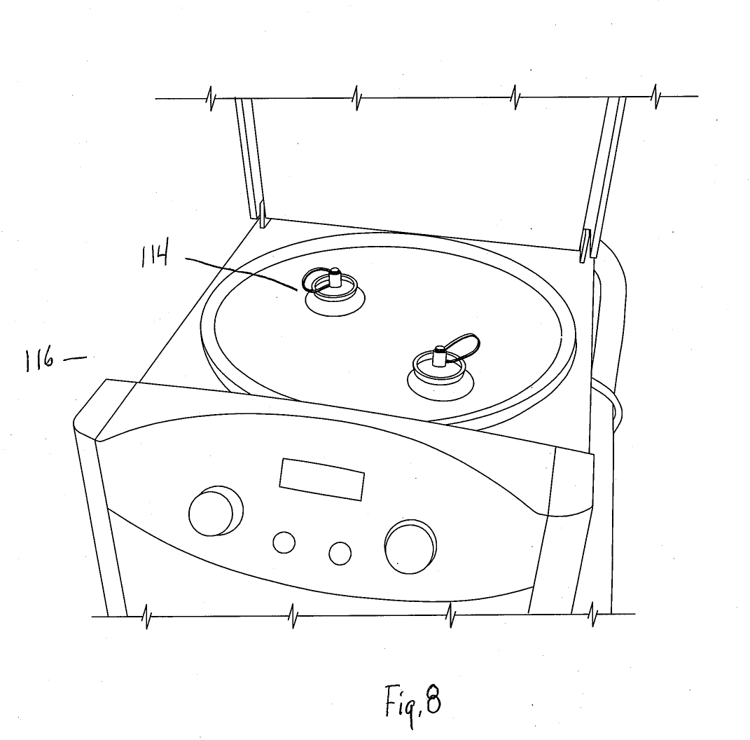

While this is being done, the bone marrow aspirate is placed by a technician in a centrifuge 116 (such as the Cyclone Concentrating System, Alliance Spine, San Antonio, Texas). to substantially separate the plasma, the red blood cells, and the buffy coat (see FIG. 8). Following centrifuging, the bone marrow concentrate plasma is substantially discarded or not drawn, the remaining fluid, the BMC 118 (see FIG. 9), now has substantially red blood cells and stem cells of the buffy coat, is transported to the bone graft or fusion assembly for saturation.

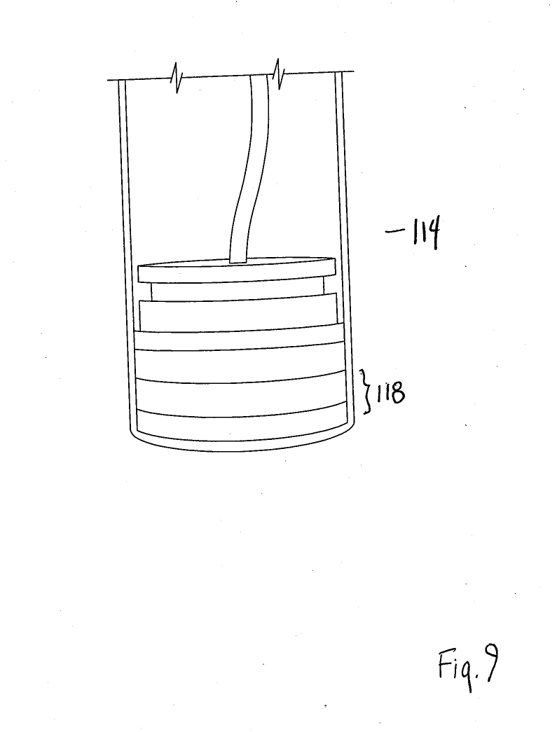

Following the step of centrifuging, a bone marrow concentrate 118 is seen at or near the bottom of a collection cup 114. FIG. 10 illustrates the surgeon using a sterile syringe to withdraw the bone marrow concentrate, seen in FIG. 10, to be partway up the tube inside the collection cup 114 with some concentrate still remaining in the bottom of the cup. This contains primarily stem cells of the buffy coat and some red blood cells for oxygen.

Prior to surgery, during the preoperative planning stage, the cage and bone graft sizes are estimated and selection of such devices is provided to the surgeon. The surgeon will then shape the bone graft, typically, in a trapezoid shape so that it is packed tightly in the center lumen of the fusion device. This fusion assembly, comprising the lumen bearing fusion device with the lumen packed with bone graft or allograft alone, is then soaked and hydrated with the enriched bone marrow concentrate for a few minutes.

A cage and bone graft assembly also called a fusion assembly 108 (see FIG. 3) has been or will now be prepared. A proper size cage 110, such as an Alamo C from Alliance Spine, San Antonio, Texas, has the center lumen 110a filled with a trapezoid shape bone graft 112, such as the Promote OsteoStrip or Promote DBM (Alliance Spine, San Antonio, Tex.). The surgeon at this step determines the proper size cage to fill the intervertebrae space. The surgeon then shapes the bone graft, typically an allograft (or autograft), to fit the lumen 110a of the selected cage 110.

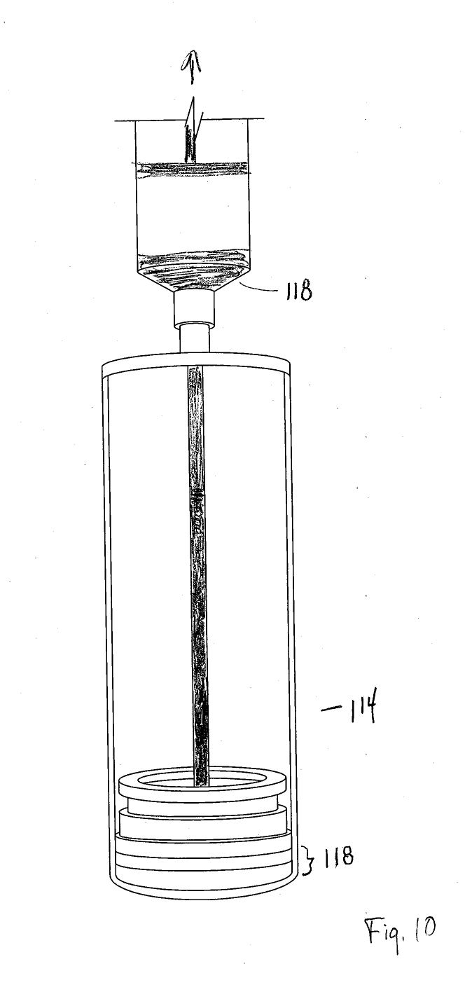

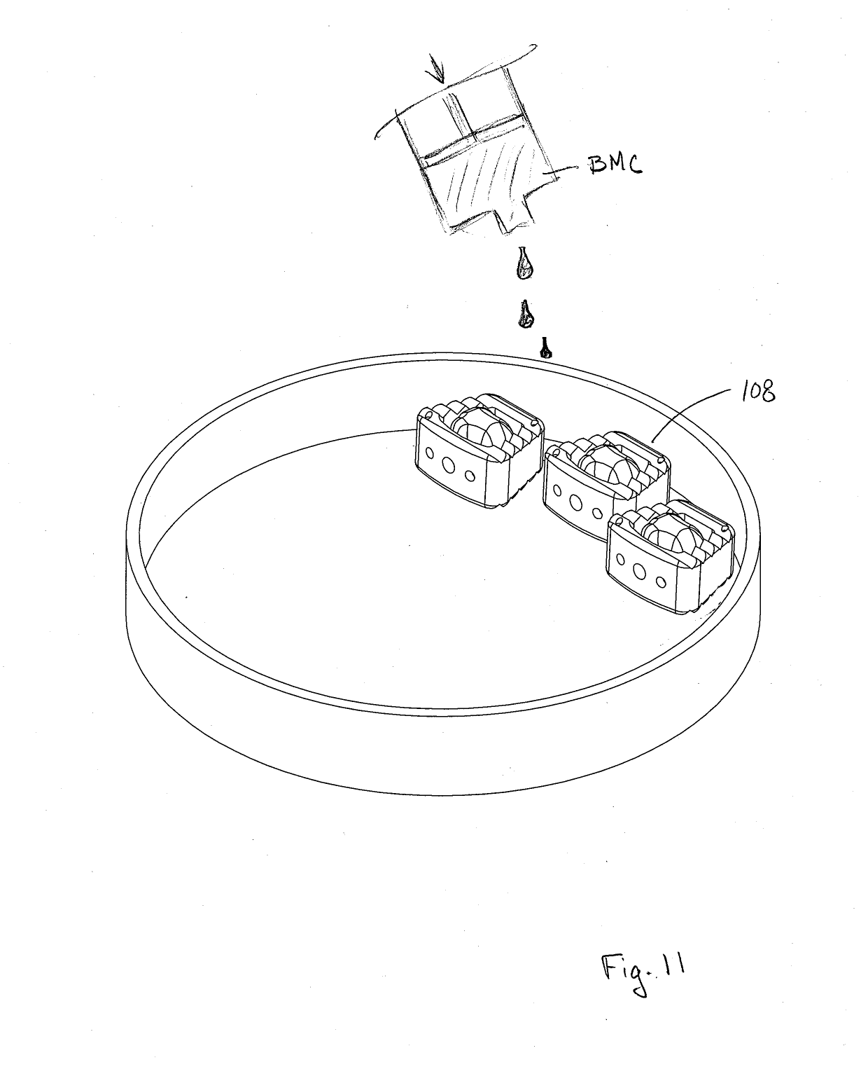

FIGS. 11 and 12 illustrate the application of the bone marrow concentrate 118 from the syringe into which it was collected (see FIG. 10) onto the fusion assemblies 108 located in a suitable cup or container, such as illustrated in FIG. 11. In FIG. 12, the soaking step is seen to be substantially complete. This may take a few minutes and does not require complete saturation of the bone graft 112, but typically at least full coverage of exposed surfaces. However, a few milliliters of BMC are usually used per assembly 108. The assembly 108 is usually turned over several times to ensure substantial wetting of all surfaces.

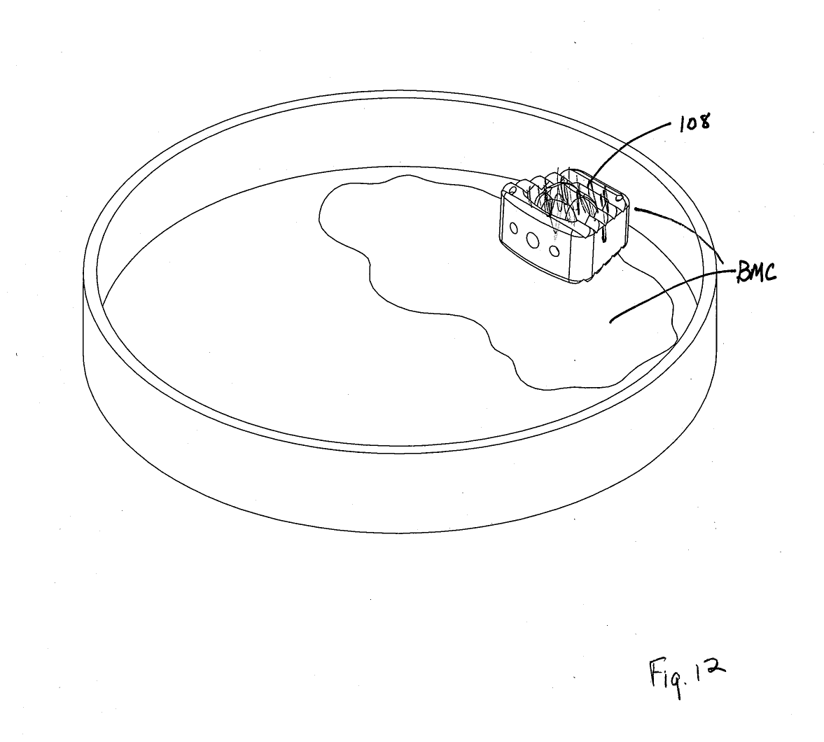

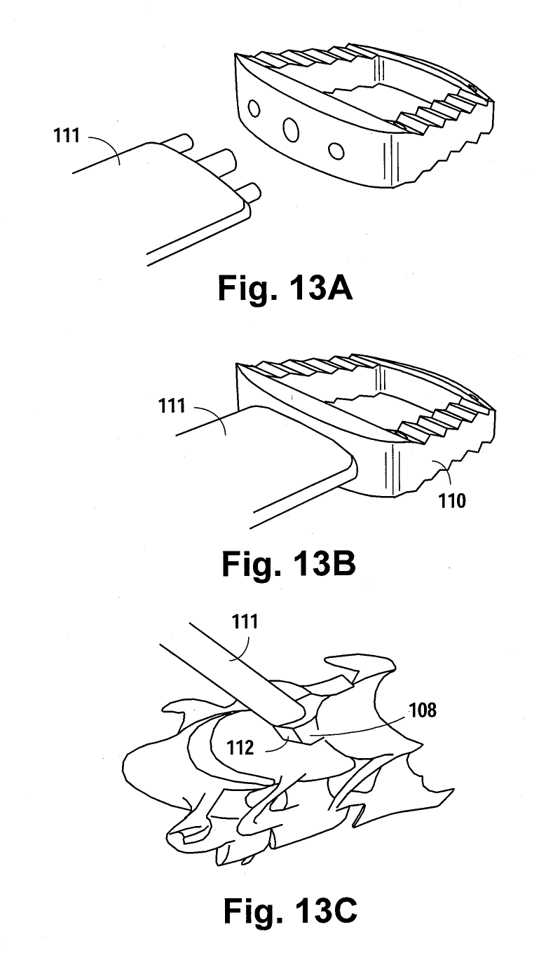

It has been found that the bone marrow concentrate from vertebral bodies, especially along the cervical area of the spine, is unusually rich in stem cells/volume aspirate ratio as compared, for example, to the stem cell/bone marrow aspirate taken from the iliac crest. The use of bone marrow concentrate will act to stimulate fusion of the bone graft to the adjacent end plate at the site where it is placed. It has been found that the bone grafts preferably should project slightly (see H, FIG. 3C) above the top and bottom surfaces of the cage, such projection, in one embodiment, in the order of about 0.5 to 3 millimeters. This will help ensure sufficient contact with the exposed upper and lower endplates of the adjacent vertebral bodies, which the assembly will fuse to. The insertion of the prepared, saturated fusion assembly 108 is done either manually or using an inserter, such as that available from Alliance Spine, AlamoC® (see FIGS. 13A-13C). To insert, attach the assembly 108 to an inserter 111 and insert in the evacuated prepared site (see FIGS. 13A, 13B, and 13C). Gentle impaction on the inserter 111 with a mallet will assist in Correctly positioning the assembly. Release the inserter by turning the handle counterclockwise to disengage the internally threaded shaft. All instruments are removed and one may verify the optimal position using an image intensifier or other suitable means. Repeat this step with additional discs, if any, and close according to conventional sterile procedure.

Qualitative and quantitative analysis and examination of about fifty patients with some examination follow-up, up to a year after the surgery, has indicated to Applicants that the fusion integrity and graft site health is at least equal to and likely better than that of use of bone marrow concentrate from the iliac crest and has the advantages of decreased trauma associated with the iliac crest bone marrow aspiration.

The step of insertion of the graft/assembly as pointed out above may be accomplished using just an appropriately shaped allograft alone, which has been soaked in the bone marrow concentrate aspirated from the anterior of the vertebral body as set forth above. Moreover, the cage alone may be inserted with any suitable graft, also subject to a soaking step.

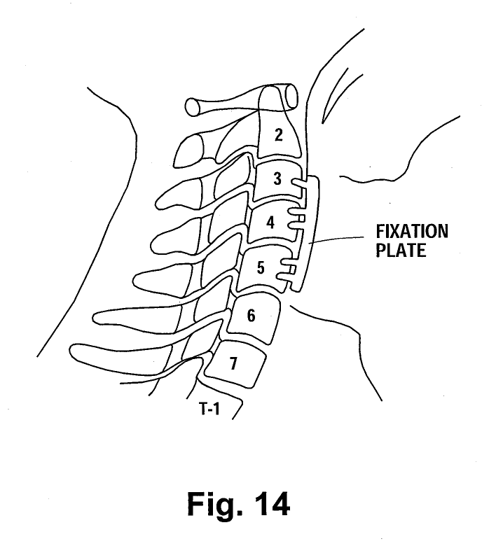

Fluoroscopy may then be used by the surgeon to determine proper positioning of the fusion assemblies. The fixation step following the insertion may be omitted in appropriate cases. A fixation plate may be provided and screwed in place in ways known in the art (see FIG. 14).

The present invention is described in terms of a preferred illustrative embodiment of specifically-described bone aspiration apparatus. Those skilled in the art will recognize that yet other alternative embodiments of such an assembly can be used in carrying out the present invention. Other aspects, features, and advantages of the present invention may be obtained from a study of this disclosure and the drawings, along with the appended claims.

1. An ACDF surgical method comprising the steps of:
providing a bone marrow aspiration needle assembly, the needle assembly including an aspiration needle and having a collecting syringe engageable therewith;
providing a bone graft fusion assembly;
identifying and exposing at least a portion of the anterior vertebrae;
inserting the aspiration needle into the anterior of an exposed cervical vertebral body at a preselected depth and a preselected orientation;
aspirating, using the bone marrow aspiration needle and collecting syringe, bone marrow from a selected cervical vertebrae body;
removing at least one cervical disc;
preparing the vertebrae end plates adjacent the removed disc;
preparing, by centrifuge, from the aspirated bone marrow, a bone marrow concentrate containing stem cells and red blood cells;
applying the bone marrow concentrate to the bone graft fusion assembly; and
inserting the bone graft fusion assembly into an inter-vertebrae space defined by the prepared end plates.

2. The method of claim 1, wherein the preselected orientation is an angular orientation of the needle axis so as to bring the needle tip closer to a selected end plates than another.

3. The method of claim 2, wherein the angular orientation of the needle is between about 5° and 40° (preferably 10 to 30°) with respect to the plane of a selected end plate of the vertebral body.

4. The method of claim 3, wherein the preselected depth is about 10 to 12 MM.

5. The method of claim 1, wherein the bone graft fusion assembly comprises a cage having a top and bottom plane with a lumen and an allograft shaped to fit substantially within the lumen.

6. The method of claim 5, wherein the allograft is shaped to extend above the top plane and below the bottom plane of the cage.

7. The method of claim 6, wherein the allograft extends above and below the cage in the range of 0.5 to 3 millimeters.

8. The method of claim 1, wherein the preselected orientation is an angular orientation of the needle axis so as to bring it closer to a selected end plates than another; wherein the angular orientation of the needle is between about 10° and 40° with respect to the plane of a selected end plate of the vertebral body; wherein the preselected depth is about 10 to 12 mm; wherein the bone graft fusion assembly comprises a cage having a top and bottom plane with a lumen and an allograft shaped to fit substantially within the lumen; and wherein the allograft is shaped to extend above the top plane and below the bottom plane of the cage.

9. The method of claim 1, further comprising the step of fixing the vertebrae adjacent the inserted bone graft.

10. The method of claim 9, wherein the preselected orientation is an angular orientation of the needle axis so as to bring it closer to a selected end plates than another; wherein the angular orientation of the needle is between about 10° and 40° with respect to the plane of a selected end plate of the vertebral body; wherein the preselected depth is about 10 to 12 mm; wherein the bone graft fusion assembly comprises a cage having a top and bottom plane with a lumen and an allograft shaped to fit substantially within the lumen; and wherein the allograft is shaped to extend above the top plane and below the bottom plane of the cage.
